(19) United States
(12) Patent Application Publication
(10) Pub. No.: US 2024/0306924 A1
(43) Pub. Date: Sep. 19, 2024

(54) ELECTRONIC DEVICE AND METHOD OF EVALUATING RISK ASSESSMENT OF CEREBROVASCULAR DISEASE

(71) Applicant: Han-Hwa Hu, Taipei City (TW)

(72) Inventors: Han-Hwa Hu, Taipei City (TW); Wen-Yung Sheng, Taipei City (TW); Men-Tzung Lo, Tainan City (TW); Hung-Yi Hsu, Taichung City (TW); Ke Han, Shenzhen (CN); Cheng-Yen Wang, Taoyuan City (TW)

(73) Assignee: Han-Hwa Hu, Taipei City (TW)

(21) Appl. No.: 18/599,174

(22) Filed: Mar. 7, 2024

(30) Foreign Application Priority Data
Mar. 17, 2023 (TW) 112109997

(51) Int. Cl. A61B 5/0205; A61B 5/00; A61B 5/02; A61B 5/021; A61B 5/026; A61B 5/08

(52) U.S. Cl. CPC A61B 5/0205; A61B 5/02028; A61B 5/021; A61B 5/026; A61B 5/0816; A61B 5/082; A61B 5/7246; A61B 5/7253; A61B 5/7275; A61B 5/7278; A61B 5/746

(57) ABSTRACT

An electronic device and a method of evaluating a risk assessment of cerebrovascular disease are disclosed. The method includes the following. A first cerebrovascular flow signal and a respiration signal are obtained. The first cerebrovascular flow signal is decomposed to obtain a first decomposed signal. Multiple first sampled signals are sampled from the first decomposed signal according to the respiration signal. A first characteristic signal is generated according to an average of the first sampled signals. It is determined whether to output a warning message according to the first characteristic signal.

### CROSS-REFERENCE TO RELATED APPLICATION

This application claims the priority benefit of Taiwan application serial no. 112109997, filed on Mar. 17, 2023. The entirety of the above-mentioned patent application is hereby incorporated by reference herein and made a part of this specification.

### BACKGROUND

### Technical Field

The disclosure relates to an electronic device and a method of evaluating a risk assessment of cerebrovascular disease.

### Description of Related Art

Early detection and treatment of cerebrovascular disease is the key to the recovery of patients. There are many and complex factors that cause the cerebrovascular disease, so the diagnosis of the cerebrovascular disease relies on many types of clinical tests. In other words, hospitals are required to spend huge equipment costs and manpower costs to more accurately evaluate a risk assessment of the cerebrovascular disease for a subject. Therefore, how to provide a more convenient method of evaluating the risk assessment of the cerebrovascular disease is one of the goals for the personnel in the art.

### SUMMARY

The disclosure provides an electronic device and a method of evaluating a risk assessment of cerebrovascular disease, which may evaluate whether a subject is at risk of suffering from the cerebrovascular disease.

An electronic device of evaluating a risk assessment of cerebrovascular disease in the disclosure includes a processor, a storage medium, and a transceiver. The storage medium stores multiple modules. The processor is coupled to the storage medium and the transceiver, and accesses and executes the modules. The modules includes a data collection module and a computation module. The data collection module obtains a first cerebrovascular flow signal and a respiration signal through the transceiver. The computation module decomposes the first cerebrovascular flow signal to obtain a first decomposed signal, samples multiple first sampled signals from the first decomposed signal according to the respiration signal, generates a first characteristic signal according to an average of the first sampled signals, and determines whether to output a warning message according to the first characteristic signal.

In an embodiment of the disclosure, the respiration signal corresponds to carbon dioxide concentration and includes a first trough and a second trough adjacent to the first trough. The computation module samples one of the first sampled signals from the first decomposed signal according to the first trough and the second trough.

In an embodiment of the disclosure, the computation module decomposes the first cerebrovascular flow signal into multiple intrinsic mode function signals according to empirical mode decomposition, and selects one of the intrinsic mode function signals as the first decomposed signal.

In an embodiment of the disclosure, the computation module obtains a respiration frequency according to the respiration signal, and selects the one of the intrinsic mode function signals that matches the respiration frequency as the first decomposed signal.

In an embodiment of the disclosure, the computation module performs Hilbert transform on the average of the first sampled signals to generate the first characteristic signal.

In an embodiment of the disclosure, the data collection module obtains a second cerebrovascular flow signal through the transceiver. The computation module generates a second characteristic signal according to the second cerebrovascular flow signal, and determines whether to output the warning message according to the first characteristic signal and the second characteristic signal.

In an embodiment of the disclosure, the first cerebrovascular flow signal includes a blood pressure signal, and the second cerebrovascular flow signal includes a blood flow velocity signal. The computation module calculates a phase difference between the first characteristic signal and the second characteristic signal, and determines to output the warning message in response to an absolute difference between the phase difference and a reference phase difference being greater than a threshold.

In an embodiment of the disclosure, the first cerebrovascular flow signal includes a blood flow velocity signal, and the second cerebrovascular flow signal includes a difference between a maximum blood flow velocity and a minimum blood flow velocity. The computation module determines to output the warning message in response to a correlation coefficient between a mean of the first characteristic signal and the second characteristic signal being less than zero.

In an embodiment of the disclosure, the first cerebrovascular flow signal includes a pulsatility index signal, and the second cerebrovascular flow signal includes a blood flow velocity signal. The computation module determines to output the warning message in response to a ratio between a first coefficient of variation of the first characteristic signal and a second coefficient of variation of the second characteristic signal being less than one.

In an embodiment of the disclosure, before the average of the first sampled signals is calculated, the computation module performs interpolation computation on at least one of the first sampled signals, so that each of the sampled signals have a same length.

A method of evaluating a risk assessment of cerebrovascular disease in the disclosure includes the following. A first cerebrovascular flow signal and a respiration signal are obtained. The first cerebrovascular flow signal is decomposed to obtain a first decomposed signal. Multiple first sampled signals are sampled from the first decomposed signal according to the respiration signal. A first characteristic signal is generated according to an average of the first sampled signals. It is determined whether to output a warning message according to the first characteristic signal.

Based on the above, the electronic device in the disclosure may determine whether the subject is at risk of suffering from the cerebrovascular disease according to the cerebrovascular flow signal of the subject. Since the cerebrovascular flow signal may be obtained non-invasive measurement, the subject does not need to endure discomfort caused by invasive measurement. When the electronic device determines that the subject is at risk of suffering from the cerebrovascular disease, the electronic device may output the warning message to prompt the subject to receive the diagnosis and treatment as soon as possible in order to obtain early treatment for the cerebrovascular disease.

### BRIEF DESCRIPTION OF THE DRAWINGS

FIG. 1 is a schematic view of an electronic device of evaluating a risk assessment of cerebrovascular disease according to an embodiment of the disclosure.

FIG. 2 is a flowchart of a method of evaluating a risk assessment of cerebrovascular disease according to an embodiment of the disclosure.

FIG. 3 is a flowchart of a method of evaluating a risk assessment of cerebrovascular disease according to another embodiment of the disclosure.

### DETAILED DESCRIPTION OF DISCLOSED EMBODIMENTS

In order for the disclosure to be more comprehensible, embodiments are described below as examples that the disclosure may be implemented accordingly. In addition, wherever possible, elements/components/steps with the same reference numerals in the drawings and embodiments represent the same or similar parts.

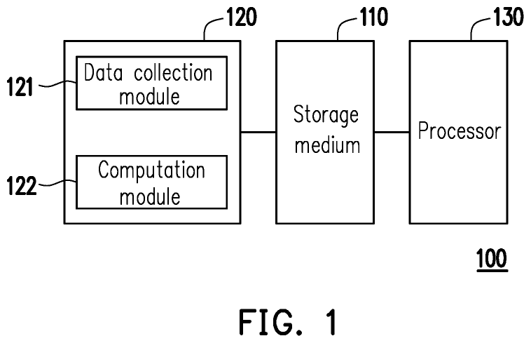

FIG. 1 is a schematic view of an electronic device 100 of evaluating a risk assessment of cerebrovascular disease according to an embodiment of the disclosure. The electronic device 100 may include a processor 110, a storage medium 120, and a transceiver 130.

The processor 110 may be a central processing unit (CPU), other programmable general-purpose or special-purpose micro control units (MCUs), a microprocessor, a digital signal processor (DSP), a programmable controller, an application specific integrated circuit (ASIC), a graphics processing unit (GPU), an image signal processor (ISP), an image processing unit (IPU), an arithmetic logic unit (ALU), a complex programmable logic device (CPLD), a field programmable gate array (FPGA), or other similar elements or a combination of the above elements. The processor 110 may be coupled to the storage medium 120 and the transceiver 130, and may access and execute multiple modules and various applications stored in the storage medium 120.

The storage medium 120 may be any type of fixed or removable random access memory (RAM), read-only memory (ROM), flash memory, hard disk drive (HDD), solid state drive (SSD), or other similar elements or a combination of the above elements, and is configured to store multiple modules or various applications that may be executed by the processor 110. In this embodiment, the storage medium 120 may store the modules including a data collection module 121 and a computation module 122, and functions thereof will be described in the following.

The transceiver 130 transmits and receives a signal in a wireless or wired manner. The transceiver 130 may further perform operations such as low noise amplification, impedance matching, mixing, up or down frequency conversion, filtering, amplification, and the like.

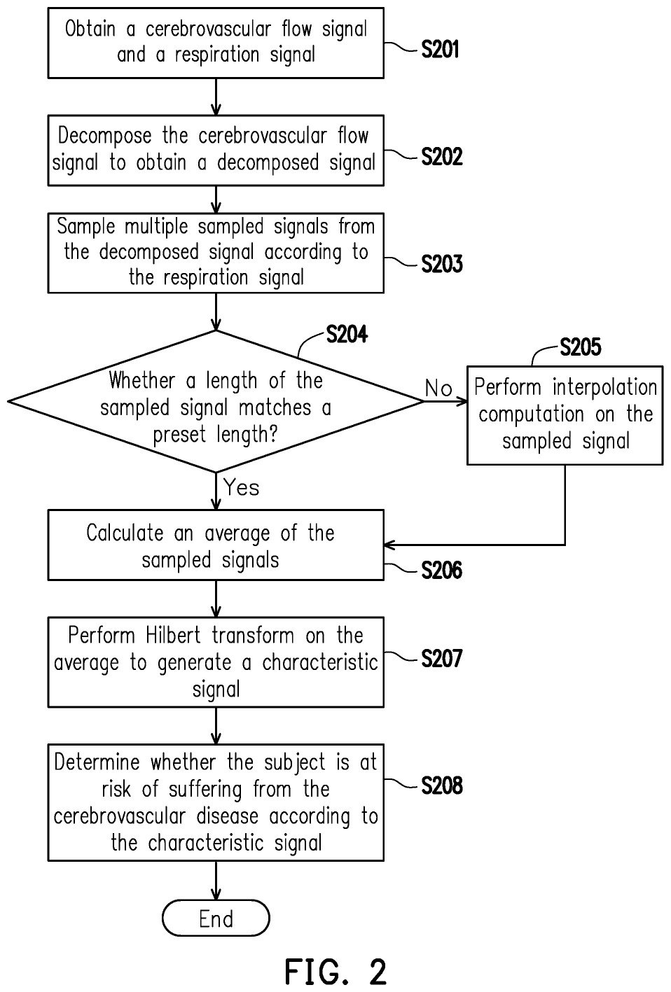

FIG. 2 is a flowchart of a method of evaluating a risk assessment of cerebrovascular disease according to an embodiment of the disclosure. The method may be implemented by the electronic device 100 shown in FIG. 1.

In step S201, the data collection module 121 may obtain one or more cerebrovascular flow signals and a respiration signal of a subject through the transceiver 130. The cerebrovascular flow signal may include but is not limited to signals such as a blood pressure (BP) signal, a blood flow velocity (BFV) signal, a maximum systolic flow velocity signal (i.e., a maximum blood flow velocity signal), or a minimum diastolic flow velocity signal (i.e., a minimum blood flow velocity signal). The blood flow velocity signal may include a BFV left (BFVL) signal or a BFV right (BFVR) signal. The respiration signal may include but is not limited to signals such as a carbon dioxide concentration signal.

During the measurement of the cerebrovascular flow signal and the respiration signal, a physician may request the subject to breathe according to a specific pattern. For example, the physician may request the subject to try to complete one respiratory cycle every 10 seconds. Starting from inhalation, cerebrovascular flow immediately increases, and concentration of carbon dioxide begins to decrease. Starting from the 5th second, exhalation begins, and a velocity of the cerebrovascular flow starts to decrease, while the concentration of carbon dioxide detected during exhalation begins to increase. At the 10th second, the cerebrovascular flow immediately increases again, and the concentration of carbon dioxide begins to decrease.

In an embodiment, the cerebrovascular flow signal may include a difference between a maximum blood flow velocity and a minimum blood flow velocity, as represented by Equation (1), DI represents the difference, S represents the maximum blood flow velocity signal, and D represents the minimum blood flow velocity signal.

$$DI = S - D … \qquad ( 1 )$$

In an embodiment, the cerebrovascular flow signal may include a pulsatility index (PI) signal, as represented by Equation (2), where PI represents the pulsatility index signal, S represents the maximum blood flow velocity signal, D represents the minimum blood flow velocity signal, and A represents an average blood flow velocity signal.

$$PI = ( S - D ) / A … \qquad ( 2 )$$

In step S202, the computation module 122 may decompose the cerebrovascular flow signal to obtain a decomposed signal. Specifically, the computation module 122 decomposes the cerebrovascular flow signal into multiple intrinsic mode function (IMF) signals respectively corresponding to different frequencies according to empirical mode decomposition (EMD). The computation module 122 may select one of the intrinsic mode function signals as the decomposed signal.

In an embodiment, the computation module 122 may obtain a respiration frequency (e.g., measuring the respiration frequency according to changes in the carbon dioxide concentration) of the subject according to the respiration signal, and select a function from multiple intrinsic mode functions that matches the respiration frequency (e.g., the intrinsic mode function signal with a frequency closest to the respiration frequency) as the decomposed signal. The intrinsic mode function selected by the computation module 122 is a component in the cerebrovascular flow signal that is related to the respiration frequency. For example, it is assumed that the intrinsic mode function signal includes the intrinsic mode function signal corresponding to a frequency of 0.5 Hz, the intrinsic mode function signal corresponding to a frequency of 1 Hz, and the intrinsic mode function signal corresponding to a frequency of 1.5 Hz. If the computation module 122 determines that the respiration frequency of the subject is 0.4 Hz, the computation module 122 will select the intrinsic mode function signal corresponding to the frequency of 0.5 Hz from the intrinsic mode function signals as the decomposed signal.

In step S203, the computation module 122 may sample multiple sampled signals from the decomposed signal according to the respiration signal. Specifically, the computation module 122 may define a sampling time as a time difference between adjacent troughs of the carbon dioxide concentration. Assuming that a first trough of the carbon dioxide concentration occurs at a first time point, and a second trough adjacent to the first trough occurs at a second time point, the computation module 122 may sample the decomposed signal between the first time point and the second time point to obtain one sampled signal.

In step S204, the computation module 122 may determine whether a length of the sampled signal matches a preset length. If the length of the sampled signal matches the preset length, the process proceeds to step S206. If the length of the sampled signal does not match the preset length, the process proceeds to step S205. For example, it is assumed that the preset length is 10 seconds. If the length of the sampled signal is 9 seconds, the computation module 122 may determine that the sampled signal does not match the preset length. If the length of the sampled signal is 10 seconds, the computation module 122 may determine that the sampled signal matches the preset length.

In step S205, the computation module 122 may perform interpolation computation on the sampled signal, so that the sampled signal matches the preset length. In other words, after steps S204 and S205 are performed on each of the sampled signals, all the sampled signals will have the same length or number of sampled points.

In step S206, the computation module 122 may calculate an average of the sampled signals. In step S207, the computation module 122 may perform Hilbert transform on the average of the sampled signals to generate a characteristic signal. In other words, the characteristic signal may be a Hilbert spectrum.

In step S208, the computation module 122 may determine whether the subject is at risk of suffering from the cerebrovascular disease according to the characteristic signal. The cerebrovascular disease is, for example, a neurovascular coupling disorder, and the disorder may cause intellectual impairment of patients. If the computation module 122 determines that the subject is at risk of suffering from the cerebrovascular disease, the computation module 122 may output a warning message through the transceiver 130 to prompt the subject to seek a medical diagnosis as soon as possible.

Specifically, it is assumed that the data collection module 121 obtains the cerebrovascular flow signals of the subject in step S201. The computation module 122 may obtain the characteristic signals corresponding to the cerebrovascular flow signals in step S207. The computation module 122 may determine whether to output the warning message of the risk assessment of the cerebrovascular disease according to the characteristic signals. If the computation module 122 determines that the subject is at higher risk of suffering from the cerebrovascular disease according to the characteristic signals, the computation module 122 may output the warning message to a terminal device (such as a computer or a smartphone) of the physician or the subject through the transceiver 130.

In an embodiment, the characteristic signals may include characteristic signals of the blood pressure signals and characteristic signals of the blood flow velocity signal. The storage medium 120 may pre-store a reference phase difference. The reference phase difference is, for example, 60 degrees. The computation module 122 may calculate a phase difference between the characteristic signals of the blood pressure signal and the characteristic signals of the blood flow velocity signal, and determine to output the warning message in response to an absolute difference between the phase difference and the reference phase difference being greater than a threshold. The greater the absolute difference, the higher the risk of the cerebrovascular disease in the subject. Accordingly, the computation module 122 may further determine a risk level of the cerebrovascular disease according to the absolute difference and output the warning message containing the risk level.

In an embodiment, the characteristic signals may include the characteristic signals of the blood flow velocity signal and characteristic signals of a difference (such as a difference DI shown in Equation (1)) between the maximum blood flow velocity and the minimum blood flow velocity. The computation module 122 may calculate a mean of the characteristic signals of the blood flow velocity signal. The computation module 122 may determine to output the warning message in response to a correlation coefficient between the mean and the characteristic signals of the difference being less than zero. The smaller the correlation coefficient, the higher the risk of the cerebrovascular disease in the subject. Accordingly, the computation module 122 may further determine the risk level of the cerebrovascular disease according to the correlation coefficient and output the warning message containing the risk level.

In an embodiment, the characteristic signals may include characteristic signals of a pulse coefficient signal (such as a pulse coefficient signal PI shown in Equation (2)) and the characteristic signals of the blood flow velocity signal. The computation module 122 may calculate a coefficient of variation (CV) of the characteristic signals of the pulse coefficient signal to obtain a first coefficient of variation, and may calculate a coefficient of variation of the characteristic signals of the blood flow velocity signal to obtain a second coefficient of variation. The computation module 122 may determine to output the warning message in response to a ratio between the first coefficient of variation and the second coefficient of variation being less than one. The smaller the ratio, the higher the risk of the cerebrovascular disease in the subject. Accordingly, the computation module 122 may further determine the risk level of the cerebrovascular disease according to the ratio and output the warning message containing the risk level.

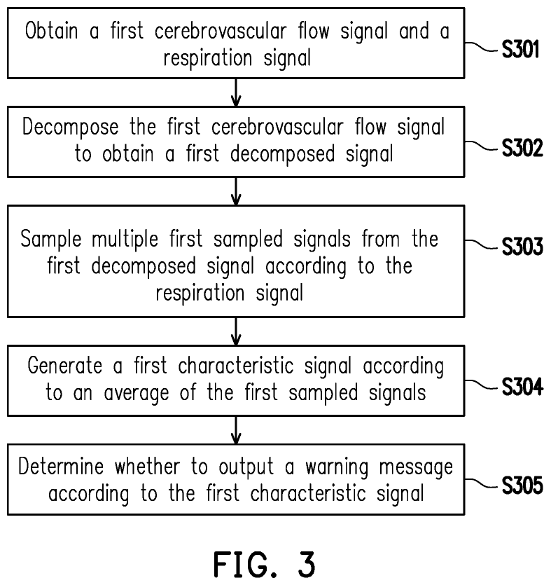

FIG. 3 is a flowchart of a method of evaluating a risk assessment of cerebrovascular disease according to another embodiment of the disclosure. The method may be implemented by the electronic device 100 shown in FIG. 1. In step S301, a first cerebrovascular flow signal and a respiration signal are obtained. In step S302, the first cerebrovascular flow signal is decomposed to obtain a first decomposed signal. In step S303, multiple first sampled signals are sampled from the first decomposed signal according to the respiration signal. In step S304, a first characteristic signal is generated according to an average of the first sampled signals. In step S305, it is determined whether to output a warning message according to the first characteristic signal.

Based on the above, the electronic device in the disclosure may determine whether the subject is at risk of suffering from the cerebrovascular disease according to the cerebrovascular flow signal of the subject. The electronic device may perform the empirical mode decomposition on the cerebrovascular flow signal to obtain the intrinsic mode function signals of the cerebrovascular flow signal. The electronic device may sample the intrinsic mode function signals that match the respiration signal according to the respiration signal to obtain the sampled signal corresponding to the respiratory cycle of the subject. The electronic device may use the Hilbert transform to obtain the characteristic signal from the sampled signal. The electronic device may analyze the characteristic signal according to preset rules to determine whether the subject is at risk of suffering from the cerebrovascular disease. Since the cerebrovascular flow signal may be obtained non-invasive measurement, the subject does not need to endure discomfort caused by invasive measurement. When the electronic device determines that the subject is at risk of suffering from the cerebrovascular disease, the electronic device may output the warning message to prompt the subject to receive the diagnosis and treatment as soon as possible in order to obtain early treatment for the cerebrovascular disease.

What is claimed is:

1. An electronic device of evaluating a risk assessment of cerebrovascular disease, comprising:
a transceiver;
a storage medium storing a plurality of modules; and
a processor coupled to the storage medium and the transceiver, and accessing and executing the modules, wherein the modules comprise:
a data collection module obtaining a first cerebrovascular flow signal and a respiration signal through the transceiver; and
a computation module decomposing the first cerebrovascular flow signal to obtain a first decomposed signal, sampling a plurality of first sampled signals from the first decomposed signal according to the respiration signal, generating a first characteristic signal according to an average of the first sampled signals, and determining whether to output a warning message according to the first characteristic signal.

2. The electronic device according to claim 1, wherein the respiration signal corresponds to carbon dioxide concentration and comprises a first trough and a second trough adjacent to the first trough, and the computation module samples one of the first sampled signals from the first decomposed signal according to the first trough and the second trough.

3. The electronic device according to claim 1, wherein the computation module decomposes the first cerebrovascular flow signal into a plurality of intrinsic mode function signals according to empirical mode decomposition, and selects one of the intrinsic mode function signals as the first decomposed signal.

4. The electronic device according to claim 3, wherein the computation module obtains a respiration frequency according to the respiration signal, and selects the one of the intrinsic mode function signals that matches the respiration frequency as the first decomposed signal.

5. The electronic device according to claim 1, wherein the computation module performs Hilbert transform on the average of the first sampled signals to generate the first characteristic signal.

6. The electronic device according to claim 1, wherein the data collection module obtains a second cerebrovascular flow signal through the transceiver, wherein the computation module generates a second characteristic signal according to the second cerebrovascular flow signal, and determines whether to output the warning message according to the first characteristic signal and the second characteristic signal.

7. The electronic device according to claim 6, wherein the first cerebrovascular flow signal comprises a blood pressure signal, and the second cerebrovascular flow signal comprises a blood flow velocity signal, wherein the computation module calculates a phase difference between the first characteristic signal and the second characteristic signal, and determines to output the warning message in response to an absolute difference between the phase difference and a reference phase difference being greater than a threshold.

8. The electronic device according to claim 6, wherein the first cerebrovascular flow signal comprises a blood flow velocity signal, and the second cerebrovascular flow signal comprises a difference between a maximum blood flow velocity and a minimum blood flow velocity, wherein the computation module determines to output the warning message in response to a correlation coefficient between a mean of the first characteristic signal and the second characteristic signal being less than zero.

9. The electronic device according to claim 6, wherein the first cerebrovascular flow signal comprises a pulsatility index signal, and the second cerebrovascular flow signal comprises a blood flow velocity signal, wherein the computation module determines to output the warning message in response to a ratio between a first coefficient of variation of the first characteristic signal and a second coefficient of variation of the second characteristic signal being less than one.

10. The electronic device according to claim 1, wherein
before the average of the first sampled signals is calculated, the computation module performs interpolation computation on at least one of the first sampled signals, so that each of the sampled signals have a same length.

11. A method of evaluating a risk assessment of cerebrovascular disease, comprising:
obtaining a first cerebrovascular flow signal and a respiration signal;
decomposing the first cerebrovascular flow signal to obtain a first decomposed signal;
sampling a plurality of first sampled signals from the first decomposed signal according to the respiration signal;
generating a first characteristic signal according to an average of the first sampled signals; and
determining whether to output a warning message according to the first characteristic signal.
